(19) United States
(12) Patent Application Publication
(10) Pub. No.: US 2021/0186925 A1
(43) Pub. Date: Jun. 24, 2021

(54) Applications Of YD1701 In Preparation Of Drugs For Treating ALDH1A3 High-Expression Tumors

(71) Applicant: ShiCang YU, Chongqing (CN)

(72) Inventors: Shicang Yu, ChongQing (CN); Jiangjie Duan, ChongQing (CN); Jun Wang, ChongQing (CN)

(73) Assignee: First Affiliated Hospital Third Military Medical University Chinese People's Liberation Army P.R Of, Chongqing (CN)

(21) Appl. No.: 16/762,497

(22) PCT Filed: Aug. 21, 2018
(86) PCT No.: PCT/CN2018/101591

§ 371 Date: May 7, 2020

(30) Foreign Application Priority Data
Nov. 7, 2017 (CN) 201711084978.0

(51) Int. Cl. A61K 31/357; A61P 35/00

(52) U.S. Cl. CPC A61K 31/357; A61P 35/00

(57) ABSTRACT

An application of YD1701 in preparation of a drug for treating ALDH1A3 high-expression tumor is disclosed. The YD1701 can be used as an ALDH1A3 inhibitor to reverse the occurrence of epithelial-mesenchymal transition (EMT) of ALDH1A3 high-expression cancer cells. It can reduce tumor invasion and metastasis to inhibit tumor progression and can prolong the overall survival time of tumor-bearring animals. YD1701 have low cytotoxicity.

### TECHNICAL FIELD

The present invention belongs to the field of biomedicine, and relates to an application of YD1701 in preparation of a drug for treating ALDH1A3 high-expression tumor.

### BACKGROUND

The cancer is a malignant tumor from epithelial tissue, is the most common type of malignant tumor, greatly harms human health and becomes the biggest killer of people in the new century. Invasion and metastasis are the main cause of death in cancer patients, and epithelial-mesenchymal transition (EMT) is one of the core cell biological events in the processes of cancer invasion and metastasis. Therefore, screening and development of small molecules that reverse the EMT is an important direction in the field of anti-cancer invasion and metastasis treatment, and has good application prospects.

Human aldehyde dehydrogenases (ALDH) family includes 19 subtypes. Different ALDH subtypes have different subcellular locations (including cell nucleus, mitochondria and cytoplasm) and different functions. The family can catalyze endogenous aldehyde to form acid to participate in the regulation of many important pathophysiological processes, comprising vision, neurotransmitter transmission, embryonic development, etc. As the research further develops, the functions of members of the ALDH family are continuously expanded at a surprising speed. Increasing results show that the members of the family play important roles in the processes of occurrence, progression, treatment resistance and recurrence of malignant tumors. Chinese patent with publication No. CN105734121A reported that the high expression of aldehyde dehydrogenase 1A3 (ALDH1A3) is closely related to the occurrence of EMT in colorectal cancer (CRC) cells; ALDH1A3 overexpression can promote the invasion and metastasis of CRC; and down-regulation of ALDH1A3 expression, inhibition of ALDH1A3 activity, or administration of ALDH1A3 overexpression effect inhibitors can completely or partially reverse the invasion and metastatic capability of CRC cancer cells. The high expression of ALDH1A3 is also found in glioma, prostate cancer, pancreatic cancer, ovarian cancer, lung cancer, liver cancer, stomach, medulloblastoma, etc., and is closely related to invasion, metastasis and poor prognosis of the above tumors. Therefore, screening ALDH1A3 inhibitors is of great significance for the treatment of ALDH1A3 high-expression tumor cells.

### SUMMARY

In view of this, the purpose of the present invention is to provide an application of YD1701 in preparation of a drug for treating ALDH1A3 high-expression tumor. The drug has low cytotoxicity and provides a new therapeutic drug for ALDH1A3 high-expression tumor.

To achieve the above purpose, the present invention provides the following technical solution:

An application of YD1701 in preparation of a drug for treating ALDH1A3 high-expression tumor.

In the present invention, the ALDH1A3 high-expression tumor is one of colorectal cancer, medulloblastoma, glioma, prostate cancer, ovarian cancer, lung cancer, gastric cancer, liver cancer or pancreatic cancer.

In the present invention, the ALDH1A3 high-expression tumor is the colorectal cancer.

In the present invention, the drug comprises YD1701 and a pharmaceutically acceptable carrier.

Preferably, the drug is any one of an injection, a capsule, a tablet and a granule.

In the present invention, an application of the YD1701 in preparation of a drug for inhibiting the invasion and metastasis of the ALDH1A3 high-expression tumor is provided.

In the present invention, an application of the YD1701 in preparation of a drug for inhibiting the progression of the ALDH1A3 high-expression tumor is provided.

In the present invention, an application of the YD1701 in preparation of a drug for prolonging the survival time of the ALDH1A3 high-expression tumor is provided.

The present invention has the beneficial effects: the present invention provides a new drug for treating ALDH1A3 high-expression tumor. The drug has low cytotoxicity, has good inhibiting effect on the ALDH1A3, reduces the invasion capability of tumor cells, inhibits tumor progression, prolongs the tumor-carrying survival time of tumor-carrying animals, can be used as a candidate drug for treatment of colorectal cancer, medulloblastoma, glioma, prostate cancer, ovarian cancer, lung cancer, gastric cancer, liver cancer and pancreatic cancer, and has important significant for the ALDH1A3 high-expression tumor.

### DESCRIPTION OF DRAWINGS

To enable the purpose, the technical solution and the beneficial effects of the present invention to be more clear, the present invention provides the following drawings for explanation:

FIG. 1 is a result diagram of screening a specific inhibitor of ALDH1A3 by molecular docking.

FIG. 2 shows compounds divided into structural clusters through fingerprint-based clustering for identification.

FIG. 3 shows a structural formula of YD1701.

FIG. 4 shows LC-MS identification results of YD1701.

FIG. 5 shows comparison results of YD1701 with DEAB, disulfiram and cimetidine.

FIG. 6 shows toxicity results of YD1701 to normal colonic mucosal epithelial NCM460 cells.

FIG. 7 is a diagram of in vitro simulation of binding of YD1701 compound and ALDH1A3 (A:3-D mode; B:2-D mode).

FIG. 8 shows cellular morphology after in vitro treatment of CRC cells by YD1701.

FIG. 9 is an expression result diagram of CDH1/E-cadherin, CDH2/N-cadherin and vimentin.

FIG. 10 is an expression result diagram of EMT transcription factors SNAI2/Slug, ZEB1.

FIG. 11 shows result diagrams of spontaneous invasion capability of different CRC cell lines and primary CRC cells after YD1701 treatment.

FIG. 12 is a result diagram of cytotoxicity on different CRC cell lines and primary CRC cells after YD1701 treatment.

FIG. 13 is a result diagram of anti-proliferation efficacy on different CRC cell lines and primary CRC cells after YD1701 treatment.

FIG. 14 is a schematic diagram of CRC subcutaneous tumor transplantation process in mice by YD1701 treatment.

FIG. 15 is a result diagram of survival time of mice of YD1701.

FIG. 16 is an effect diagram of YD1701 treatment for subcutaneous tumor transplantation in mice.

FIG. 17 is a schematic diagram of YD1701 and 5-FU treatment processes for orthotopic CRC transplanted tumors in mice.

FIG. 18 is an observation result diagram of in-vivo imaging of animals on the 20th day after colons of mice are implanted with tumor micro-tissue blocks orthotopically by observation with an in-vivo imaging system.

FIG. 19 is a result diagram of total survival time of orthotopic CRC tumor-bearring mice after YD1701 treatment.

FIG. 20 is an effect diagram of YD1701 treatment for orthotopic transplanted tumors in mice, comprising tumor in situ, liver and pulmonary metastasis.

FIG. 21 is an inhibition result diagram of YD1701 on medulloblastoma, prostatic cancer, lung cancer, ovarian cancer, gastric cancer and liver cancer invasion phenotypes.

### DETAILED DESCRIPTION

Preferred embodiments of the present invention will be described below in detail in combination with drawings.

### Embodiment 1 ALDH1A3 Inhibitor Screening

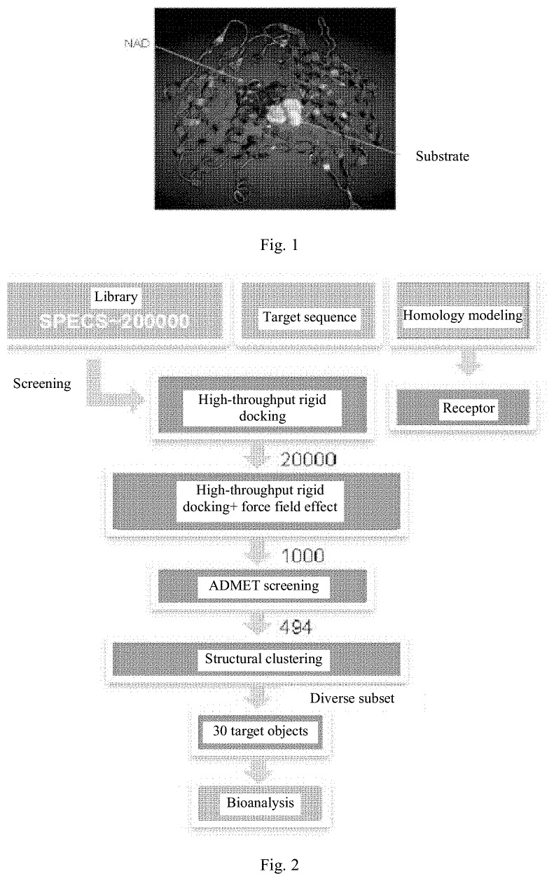

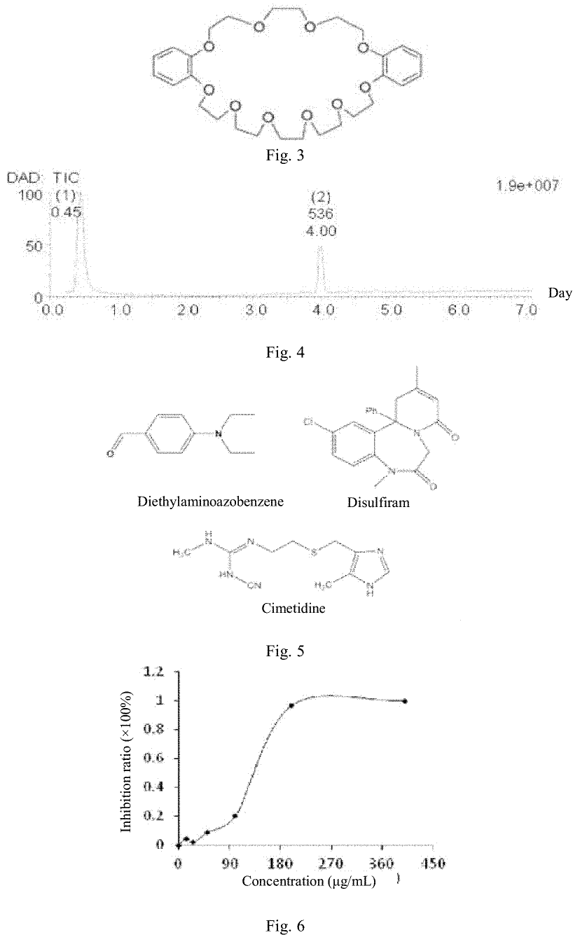

A specific inhibitor of ALDH1A3 is screened by molecular docking (FIG. 1). Before the molecular docking, firstly, salt ions are removed, only a largest molecular fragment is reserved and tautomers and protonation are enumerated to prepare a target library (>200,000 compounds). Then, a 3-D structure is generated by energy minimization. Next, with high-throughput docking, the first 20,000 compounds are further flexibly docked, and the attitude of the docking is refined with a force field. Then, the ADMET descriptors of the first 1000 compounds are calculated, and the compounds with poor ADMET properties are filtered. In the ADMET filtering process, all compounds with log P>6 or alarm number>3 are filtered. The 494 selected compounds are divided into structural clusters through fingerprint-based clustering, and 30 compounds are finally identified. The specific process is shown in FIG. 2. In the 30 compounds, based on the toxic effect on normal colonic mucosal epithelial cells, the compound YD1701 with the minimum toxic reaction is selected. The molecular weight of YD1701 molecular formula=536.63. A structural formula is shown in FIG. 3. Then, the YD1701 compound is subjected to LC-MS identification, and results are shown in FIG. 4. The identified YD1701 compound is compared with a known ALDH inhibitor. The results show that the screened YD1701 compounds is different from the known ALDH inhibitor, such as DEAB, disulfiram and cimetidine (FIG. 5), indicating that the YD1701 compound is a new ALDH inhibitor. It is predicted that the YD1701 compound may have a therapeutic effect on the ALDH high-expression tumor.

### Embodiment 2 YD1701 Cytotoxicity Experiment

To clarify whether the YD1701 compound can be used for treatment of ALDH1A3 high-expression tumor, the cytotoxicity of the YD1701 compound on normal cells needs to be researched. The normal colonic mucosal epithelial NCM460 cell line (purchased from the American Type Culture Collection, ATCC) is expanded and cultured until the cells are in good condition. After each cell is digested with trypsin and digested with neutralizing trypsin, the cells are washed once with sterile PBS and then are counted. The concentration of each cell is adjusted to 5×10⁴/ml with a complete medium, and then the cell suspension of the adjusted concentration is placed in a sterile 10 cm cell culture dish. The cell culture dish is gently shaken around to uniformly mix the cells. The cell suspension is evenly added to a 96-well plate by using a pipette, with each well having 100 μL, and cultured at 37° C. overnight. After the cells adhere to the wall, YD1701 compounds with the concentrations of (0 μg/mL, 0.04 μg/mL, 0.2 μg/mL, 1 μg/mL, 5 μg/mL, 25 μg/mL, 125 μg/mL and 250 μg/mL) are added. No cell suspension is added to the outer ring of the 96-well plate, and a sterile PBS is added to prevent the culture medium from evaporating. Then, the solution is changed with the pipette, and the complete medium containing the corresponding concentration of the YD1701 compound is added to each well. The corresponding different concentrations of drug-containing culture media are also added to the cell-free wells as cell-free blank control. 100 μL of drug-containing complete medium is added to each well, and at least 5 duplicate wells are made for each concentration, and cultured at 37° C. After the drug treatment is completed, 10 μL of CCK-8 solution is added to each well to continue the culturing in a cell incubator for 1 hour. The absorbance is measured at 450 nm. The experimental results are expressed by cell survival rate: cell survival rate (%)=(drug treatment group-corresponding drug concentration of blank control)/(non-drug treatment group-blank control)×100%. The results are shown in FIG. 6. The results show that the YD1701 compound has low toxicity to normal colonic mucosal epithelial NCM460 cells, IC₅₀>100 μg/mL.

### Embodiment 3 Inhibition of YD1701 Compound on Colorectal Cancer (CRC) Cells

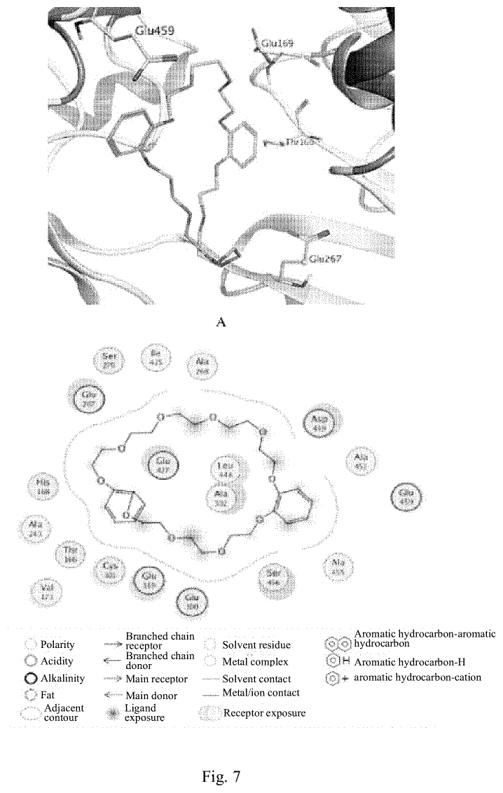

Because the high expression of acetaldehyde dehydrogenase 1A3 (ALDH1A3) is closely related to the occurrence of EMT in colorectal cancer (CRC) cells, in order to research whether the YD1701 compound promotes the mesenchymal-epithelial transition (MET) of the CRC cells, the binding degree of the YD1701 compound and ALDH1A3 is subjected to in vitro simulation, and results are shown in FIG. 7. The results show that the YD1701 compound and ALDH1A3 are in a pocket binding mode, and it is predicted that the YD1701 compound may have a good inhibiting effect on ALDH1A3.

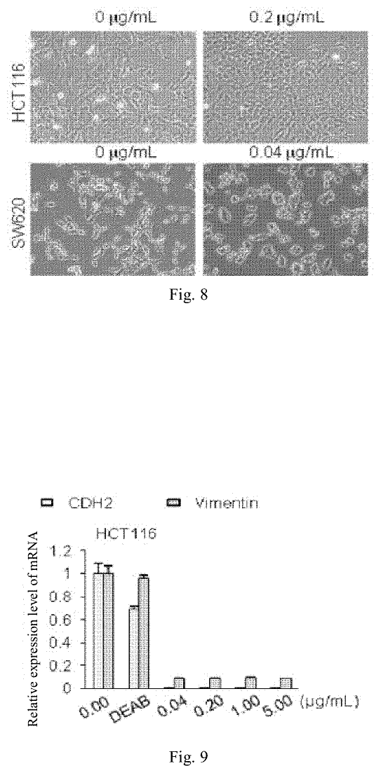

To assess whether the YD1701 can inhibit the function of ALDH1A3 and promote the occurrence of MET in the CRC cells, the CRC cells (HCT116, HT29, SW480, SW620 and colon cancer primary cells CRC1) are treated by in vitro using (0 μg/mL, 0.04 μg/mL, 0.2 μg/mL, 1 μg/mL, 5 μg/mL and 25 μg/mL) YD1701. Then, the cell morphology is observed, and the results are shown in FIG. 8. The results show that the CRC cells have obvious MET-related morphological change, and the cells are changed from spindle cells to elliptical or polygonal cells.

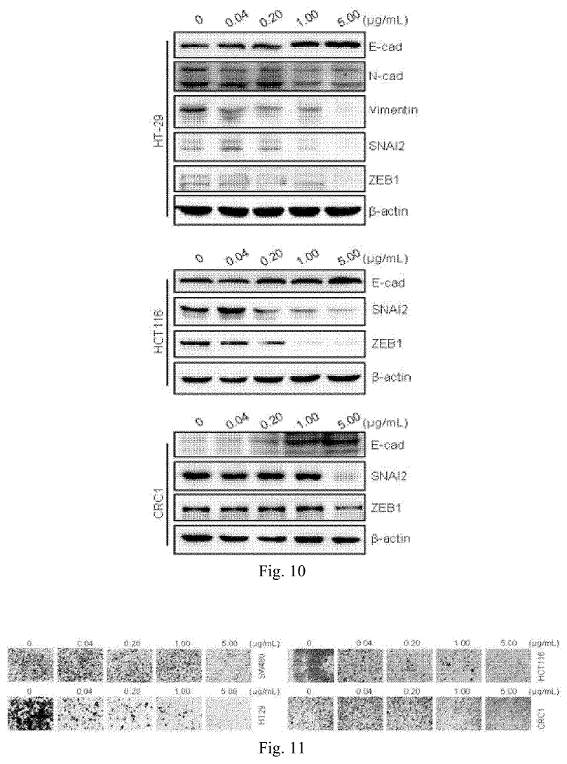

Epithelial marker E-cadherin, mesenchymal marker CDH2/N-cadherin, vimentin and EMT transcription factors SNAI2/Slug, ZEB1 are detected. Results are shown in FIG. 9 and FIG. 10. The results show that the epithelial marker (E-cadherin) is up-regulated, and the expressions of the mesenchymal markers (CDH2/N-cadherin, vimentin) and EMT transcription factors (SNAI2/Slug, ZEB1) are obviously down-regulated.

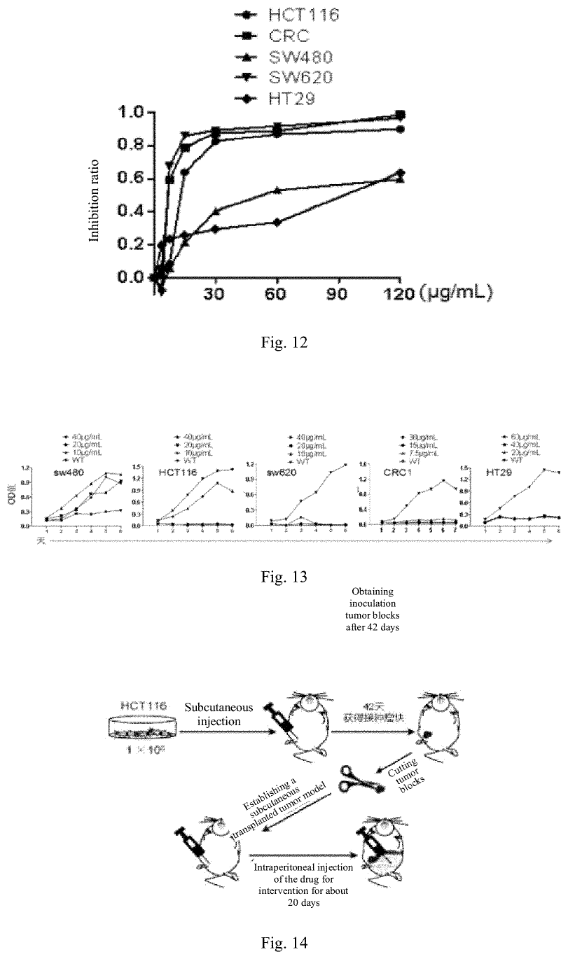

Then, the spontaneous invasion capability of the YD1701 compound treatment on different CRC cell lines (HCT116, HT29 and SW480) and primary CRC cells (CRC1) is investigated. Matrigel transwell determination results are shown in FIG. 11. The results show that after YD1701 (<0.04 μg/mL) is added, the spontaneous invasion capability of different CRC cell lines and primary CRC cells is obviously reduced. Next, the cytotoxicity and anti-proliferation efficacy of the YD1701 compound treatment on different CRC cell lines (HCT116, HT29, SW480 and SW620) and the primary CRC cells (CRC1) are investigated. CCK-8 determination results are shown in FIG. 12 and FIG. 13. The results show that the cytotoxicity and the anti-proliferation efficacy of the YD1701 compound on different CRC cell lines and the primary CRC cells are not obvious.

### Embodiment 4 Assessment of Therapeutic Efficacy of YD1701 on Rectal Cancer

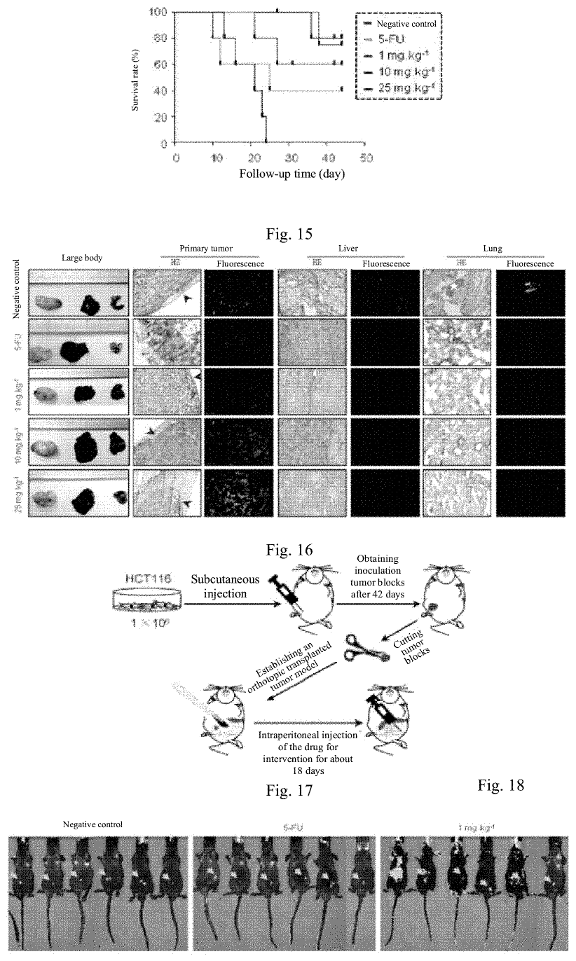

To assess the therapeutic efficacy of the YD1701, a CRC subcutaneous tumor transplantation experiment in mice is conducted, and an experimental process is shown in FIG. 14. After inoculation, when the tumor volume reaches about 100 mm³, the mice are divided into 5 groups. YD1701 (high dose, 25 mg·kg⁻¹; medium dose, 10 mg·kg⁻¹; low dose, 1 mg·kg⁻¹), normal saline+DMSO (negative control), and 5-FU (positive control, 10 mg·kg⁻¹) are respectively added through intraperitoneal injection for treatment for two weeks; the progression of CRC subcutaneous tumor transplantation in the mice is observed; and the survival time of the mice is counted, as shown in FIG. 15. The results show that no significant difference exists in the tumor volume, but the YD1701 treatment significantly prolongs the overall survival time of the tumor-carrying mice. Detection of liver and lung metastasis in the tumor transplantation mice is shown in FIG. 16. The results show that liver and lung metastases are detected in some mice of the negative control group.

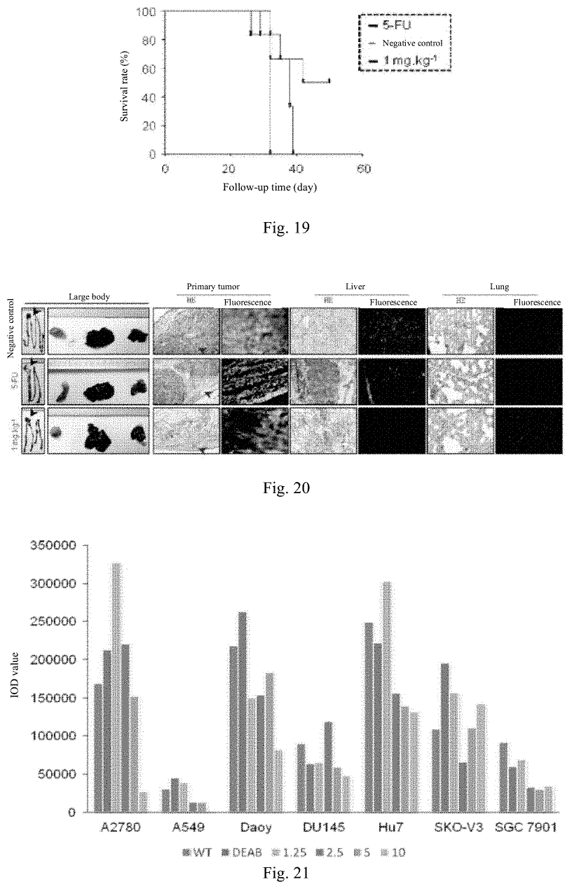

To further assess the therapeutic potential of the YD1701, the inhibiting effects of the YD1701 and 5-FU(5-fluorouracil) on orthotopic CRC transplanted tumors in the mice are compared, and the experimental process is shown in FIG. 17. On the 20th day after the colons of mice of YD1701 are implanted with tumor micro-tissue blocks orthotopically, and the formation of the orthotopic transplanted tumors is determined by an in-vivo imaging system. The results are shown in FIG. 18. The results show that all tested mice form tumors of uniform size in the colons. Then, the determined mice with the orthotopic transplanted tumors are divided into three groups and subjected to intraperitoneal injection of YD1701 (1 mg·kg⁻¹), normal saline+DMSO and 5-FU (10 mg·kg⁻¹) for treatment for two weeks. The total survival time of the three groups of mice with the orthotopic transplanted tumors is compared. The results are shown in FIG. 19. The results show that the YD1701 treatment group significantly prolongs the total survival time of the orthotopic CRC tumor-carrying mice compared with the normal saline+DMSO and 5-FU (10 mg·kg⁻¹) treatment group mice, and no metastasis appears in the mice of the YD1701 treatment group. However, in some mice of the control groups, significant liver metastasis can be observed (FIG. 20), and the volume sizes of the tumors in situ in the colons of the three groups of mice have no obvious difference (FIG. 20).

The above results show that the ALDH1A3-specific inhibitor YD1701 can promote the MET phenotype of the CRC cells, inhibit the progression of human CRC, and reduce the metastasis of CRC.

### Embodiment 5 Assessment of Therapeutic Efficacy of YD1701 on Other Cancers

In addition to the colorectal cancer, the high expression of ALDH1A3 is also found in high-grade glioma, prostate cancer, pancreatic cancer, ovarian cancer, lung cancer, gastric cancer, liver cancer and medulloblastoma, and is closely related to the invasion, metastasis and prognosis of the above tumors . Therefore, according to the method of embodiment 3, the spontaneous invasion capability of the YD1701 for medulloblastoma (Daoy), prostate cancer (DU145), lung cancer (A549), ovarian cancer (SKO-V3, A2780), liver cancer (Hu-7) and gastric cancer (SGC-7901) is respectively assessed. The results are shown in FIG. 21. The results show that the invasion capability of the above tumor cells after YD1701 treatment is significantly reduced. The above results show that the YD1701 has application prospects for the treatment of lung cancer, gastric cancer, liver cancer, prostate cancer, ovarian cancer and medulloblastoma.

Finally, it should be noted that the above preferred embodiments are only used for describing, rather than limiting the technical solution of the present invention. Although the present invention is already described in detail through the above preferred embodiments, those skilled in the art shall understand that various changes in form and detail can be made to the present invention without departing from the scope defined by claims of the present invention.

1. An application of YD1701 in preparation of a drug for treating ALDH1A3 high-expression tumor.

2. The application according to claim 1, characterized in that the ALDH1A3 high-expression tumor is one of colorectal cancer, medulloblastoma, glioma, prostate cancer, ovarian cancer, lung cancer, gastric cancer, liver cancer or pancreatic cancer.

3. The application according to claim 1, characterized in that the ALDH1A3 high-expression tumor is the colorectal cancer.

4. The application according to claim 1, characterized in that the drug comprises YD1701 and a pharmaceutically acceptable carrier.

5. The application according to claim 1, characterized in that the drug is any one of an injection, a capsule, a tablet and a granule.

6. The application according to claim 1, characterized in an application of the YD1701 in preparation of a drug for inhibiting the invasion and metastasis of the ALDH1A3 high-expression tumor.

7. The application according to claim 1, characterized in an application of the YD1701 in preparation of a drug for inhibiting the progression of the ALDH1A3 high-expression tumor.

8. The application according to claim 1, characterized in an application of the YD1701 in preparation of a drug for prolonging the survival time of the ALDH1A3 high-expression tumor.
